(19) United States
(12) Patent Application Publication
(10) Pub. No.: US 2014/0199586 A1
(43) Pub. Date: Jul. 17, 2014

(54) APPARATUS FOR EVAPORATING LIQUID HYDROCARBON COMPOUNDS OR OF LIQUIDS IN WHICH HYDROCARBON COMPOUNDS ARE CONTAINED AS WELL AS USE OF SAME

(71) Applicants: Manuela Breite, Dresden (DE); Matthias Jahn, Dresden (DE); Adrian Goldberg, Dresden (DE)

(72) Inventors: Manuela Breite, Dresden (DE); Matthias Jahn, Dresden (DE); Adrian Goldberg, Dresden (DE)

(73) Assignee: SUEDZUCKER AG MANNHEIM/OCHSENFURT, Mannheim (DE)

(21) Appl. No.: 14/119,630

(22) PCT Filed: May 2, 2012
(86) PCT No.: PCT/EP2012/057967

§ 371 Date: Feb. 12, 2014

(30) Foreign Application Priority Data
May 23, 2011 (DE) 102011102224.8

(51) Int. Cl. H01M 8/06; C07C 29/80

(52) U.S. Cl. CPC H01M 8/0662; C07C 29/80
USPC 429/210

(57) ABSTRACT

The invention relates to an apparatus for evaporating liquid hydrocarbon compounds or liquids in which at least one hydrocarbon compound is contained and to a use of same. It is the object of the invention to provide an apparatus for evaporating hydrocarbon compounds or liquids in which such compounds are contained, wherein the vapor formed can be provided with a very small pressure difference and in a suitable consistency. A heating apparatus with which a heating can be achieved above the boiling temperature of the respective hydrocarbon compound or of a liquid is present at the apparatus. The hydrocarbon compound or the liquid flows through at least one hollow space which is formed in a body or in a structure and the body or the structure is formed from a ceramic material which is inert for the respective hydrocarbon compound and/or for chemical compounds or chemical elements contained in the liquid.

The invention relates to an apparatus for evaporating liquid hydrocarbon compounds or of liquids in which at least one hydrocarbon compound is contained and to a use of same.

A number of differently configured evaporators are used for the evaporation of liquids. The heating of the liquid to be evaporated takes place using different heating devices in this respect. Burners, heat exchangers or also electrical heating devices are preferred in this respect.

It is generally problematic in this respect to make the vapor formed after the boiling available for a further use without larger pressure fluctuations (pulsation-free). The large-volume vessels which are usually used and through which the vapor formed can be led for a pressure compensation have the disadvantage that not only a reduction in the flow speed and in the pressure of the vapor occurs, but also its temperature is lowered. The vapor thereby has to be formed at an elevated temperature, which lies considerably above the boiling temperature, to avoid too early a condensation.

The use of valves for a pressure compensation of the vapor also only has a restricted effect. Valves, which can also be adjustable, are associated with increased costs, on the one hand, and they are subject to increased wear, on the other hand. The high costs for the correspondingly temperature-resistant valves are a substantial disadvantage in this respect.

A further problem is given by the hydrocarbon compounds to be evaporated or by chemical compounds contained in the liquids to be evaporated. They may have a chemically aggressive effect, may cause corrosion or may react chemically at the temperatures required for the evaporation.

The named problems and disadvantages in particular have an effect in the evaporation of ethanol or of ethanol-water mixtures and in this respect in particular when ethanol is to be used as a fuel for operation in high-temperature fuel cells. This effect is amplified since ethanol is not or may not be used in a chemically pure form for most applications. As a rule, for instance, denaturing agents are contained, for instance, which are intended to prevent direction consumption. Such substances can, however, react chemically in the evaporation, which results in disadvantages. It is moreover disadvantageous that the boiling temperature also deviates from the boiling temperature of the hydrocarbon compound actually to be evaporated and in particular of ethanol.

It is therefore the object of the invention to provide an apparatus for evaporating hydrocarbon compounds or liquids in which such compounds are contained, wherein the vapor formed can be provided with a very small pressure difference and in a suitable consistency. In addition, no chemical reaction should be able to occur in the evaporation and in particular with a material with which the apparatus is formed.

This object is achieved in accordance with the invention by an apparatus having the features of claim 1. A use is given by claim 12. Advantageous embodiments of the invention can be realized using features designated in subordinate claims.

A heating apparatus with which a heating can be achieved above the boiling temperature of the respective hydrocarbon compound or of a liquid is present at the apparatus in accordance with the invention. The hydrocarbon compound or liquid flows through at least one hollow space which is formed in a body or in a structure and is heated to a temperature above the boiling temperature in so doing. The body or the structure is formed from a ceramic material which is inert for the respective hydrocarbon compound and/or for chemical compounds or chemical elements contained in the liquid.

Inert is to be understood in this respect such that no chemical reaction can occur with the material and with the respective hydrocarbon compound as well as with any component contained in the liquid. They are in particular chemical materials in which no cobalt and no chromium are contained.

The hollow spaces within the body or within the structure can be at least one passage, pores in an open-pore ceramic foam body or free spaces between ceramic fibers from which a structure is formed.

In an alternative in accordance with the invention, the hydrocarbon compound or the liquid can flow through a passage which is formed in a ceramic body. There is, however, also the possibility of allowing the flow to flow through a plurality of passages formed within the body and in so doing to achieve the heating up to the evaporation. In this respect, a division of the total liquid flow introduced via at least one inlet can take place within the body.

The individual passages can in this respect have different lengths and solely or additionally thereto can also have different free cross-sectional surfaces. A different time is thereby required for the flowing through of the individual passages until the vapor formed can leave the apparatus for a following use, whereby a further reduction in the pressure fluctuations, which occur in time, of the formed vapor which is discharged can be achieved.

One or more passages can be guided through the body in a meandering manner. The construction size can thereby be reduced, in particular under the aspect of heating.

The outlet of one or more passages can have a tapering of the size of the free cross-sectional surface which is adjoined by a region having an enlarging free cross-sectional surface in the flow direction of the vapor formed. The flow speed of the vapor formed is thus increased in the region of the tapering, whereby the discharge can be facilitated and a deposition formation in the passage or in the passages can be avoided. The flow speed and the pressure and possibly pressure fluctuations of the vapor formed which occur over time can thus be further compensated and balanced using the adjoining widening and the free cross-section which is larger there and through which the vapor formed can flow.

The free cross-sectional area in the region of the tapering should be at least 10% smaller than the free cross-sectional surface of a passage in front of it. The region of the widening which is arranged subsequent to the tapering should be configured as conically widening in the flow direction of the vapor.

Otherwise, passages can be formed from the inlet of the hydrocarbon compound or liquid up to the tapering with a constant free cross-sectional surface.

Those bodies made of ceramic materials which can be used in the invention can be produced simply, flexibly and inexpensively from laminates/films. The individual layers/laminates/films can be brought into the respectively desired shape before the actual sintering. In this respect, regions can be cut out of them (e.g. by laser cutting) or can be removed in another way. The layers/laminates/films are then stacked over one another and, where necessary, also sintered under the effect of compressive force in a technology known per se so that the body is formed after the sintering from the layers/laminates/films which are connected to one another with material continuity and in this respect also leaktight for liquids and gases. The channel or channels is/are then formed in a multilayer design.

The likewise known LTCC or HTCC ceramic materials can be used for this purpose.

There is also the possibility in the invention that at least one further passage is formed in the body through which a hot medium can be conducted, preferably in cross-flow or in counter-flow, with which a heating can be achieved to a temperature at least above the boiling temperature of the hydrocarbon compound. Such a passage for hot medium can then be conducted through the body next to, preferably at least regionally parallel to, one or more passages so that the heating of the hydrocarbon compound or of the liquid can be achieved by heat exchange.

In this respect, process heat loss and in particular hot exhaust gas can be used as the hot medium. The heat loss of high-temperature fuel cells or of an ignition burner for such cells can thus be used, for example, whose hot exhaust gas can then preferably be used for the evaporation. The total efficiency of a system, for example of an SOFC, can thereby also be increased using an apparatus in accordance with the invention (by saving balance-of-plant energy).

In an embodiment of the invention having a ceramic foam body, the porosity and/or the pore size within the foam body should be increased in the flow direction of the vapor formed. A positive effect can thereby likewise be applied to the flow speed of the vapor formed and of the pressure up to the discharge from the apparatus and the pressure differences which occur over time after the discharge of the vapor can thereby be further reduced. The change in the porosity and/or pore size can in this respect take place continuously or in an at least two-fold stage.

The manufacture of suitable foam bodies from ceramic materials forms part of the prior art. In this respect, a porous base body from organic material is coated with a mixture of ceramic powder and binder at the surface, and in particular also in the interior, of the foam. On a heat treatment, the organic components are largely removed as a result of pyrolysis and the ceramic powder is then sintered so that a corresponding ceramic foam body is obtained.

A homogeneous foam body used in the apparatus in accordance with the invention should thus have a pore density of at least 15 ppi, preferably 20 ppi, and a porosity of 80% to 95%, preferably of 80% to 90%.

With foam bodies having a foam structure varying in the flow direction, a region can first be flowed through which has a pore density of at least 15 ppi, preferably 20 ppi, and which is adjoined by a region having a larger pore size. This region can have a pore density of 30 ppi and can then make up at least half, preferably three-quarters of the flowed-through path length through the foam body.

In a further alternative for the invention, a structure formed using ceramic fibers can also be used in which the hollow spaces are formed with free spaces between the fibers. The ceramic fibers can in this respect form the structure as a non-crimp fabric, a knitted fabric, a woven fabric or a meshwork. There is also the possibility of connecting the fibers to one another with material continuity. Green fibers which have not yet been sintered can be brought into the desired form for this purpose and can then be connected to one another at points at the contact sites via sintering bridges in a heat treatment resulting in sintering.

Ceramic materials can be used for bodies or structures usable in the invention which are selected from SiC, Si₃N₄, WC, AlN, TiN and molybdenum silicide.

On the use of electrically conductive ceramic materials such as SiC (SSiC and CSiC are preferred), TiN, WC or molybdenum silicide, there is the possibility of a heating by direct connection to an electrical voltage source to achieve the heating directly with the body or the structure which results in the evaporation. The body or the structure in this respect forms an electrical resistance heating source. These ceramic materials are also very suitable due to their good thermal conductivity. The body or the structure in this respect forms a heating element.

There is, however, also the possibility of conducting electrically conductive elements such as metal wires through a body or a structure or to insert the body or the structure into an electrically conductive element and to utilize them with an electrical connector as a heating element. Analog to this, however, at least one tube can also be provided around or at the body or structure through which tube a hot medium flows to heat the hydrocarbon compound or the liquid up to and above the boiling temperature by heat exchange. The body or a structure can also be arranged in a vessel through which a hot medium flows for heating. The heat loss of exhaust gas from a process can also be used here.

A combination of an electrical resistance heating element with a heating element in which the heating takes place by heat exchange is also possible.

The liquid hydrocarbon compound or the liquid can be supplied to the apparatus from a vessel which is arranged in the vertical direction such that a conveying of the hydrocarbon compound or of the liquid can be achieved solely as a consequence of the acting gravitational force in the apparatus. The entry should preferably take place vertically downwardly or in the vertically lower region of the apparatus and the removal should accordingly take place vertically upwardly or in the vertical upward region.

The invention will be explained in more detail in the following with reference to examples.

There are shown:

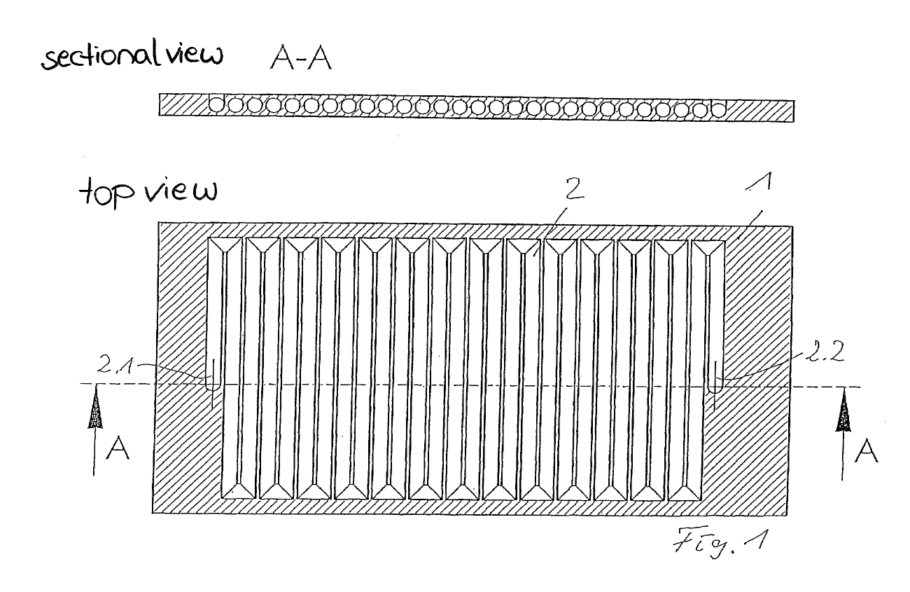

FIG. 1 a sectional representation through an example with a passage which is guided through a body in a meandering manner;

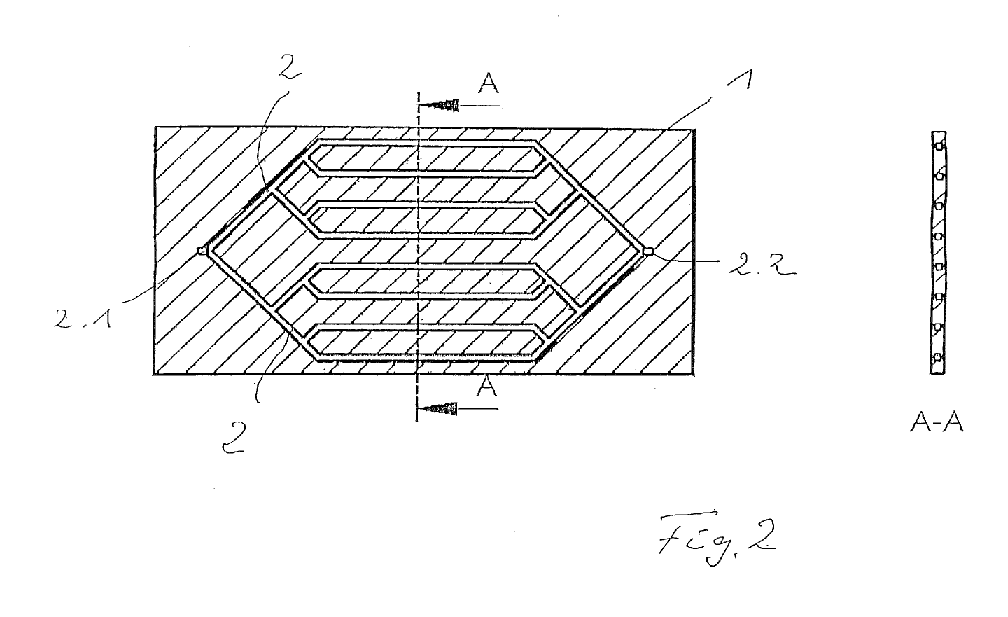

FIG. 2 a sectional representation through a body with branched

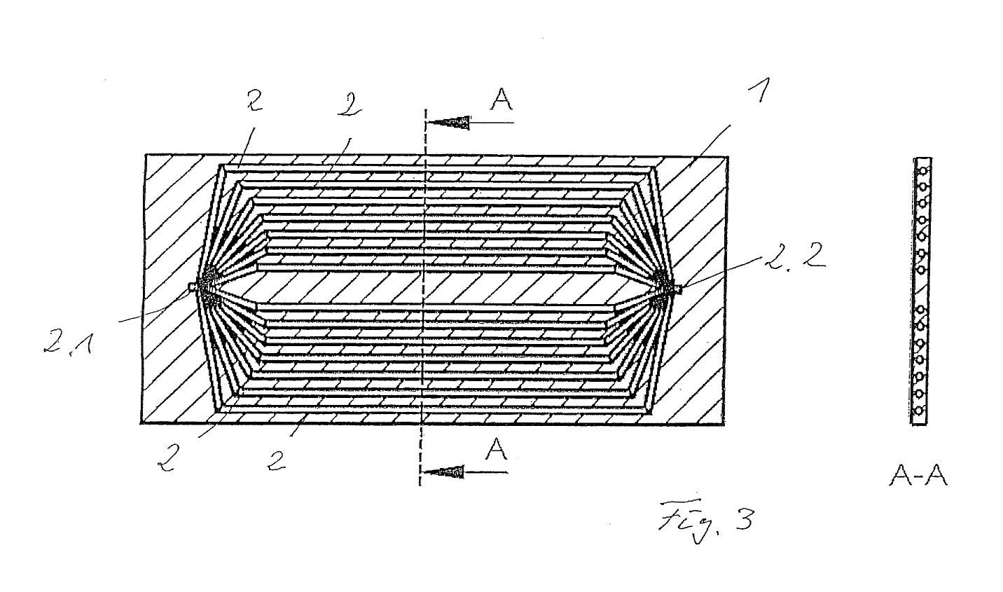

FIG. 3 a sectional representation through a body with a plurality of passages; and

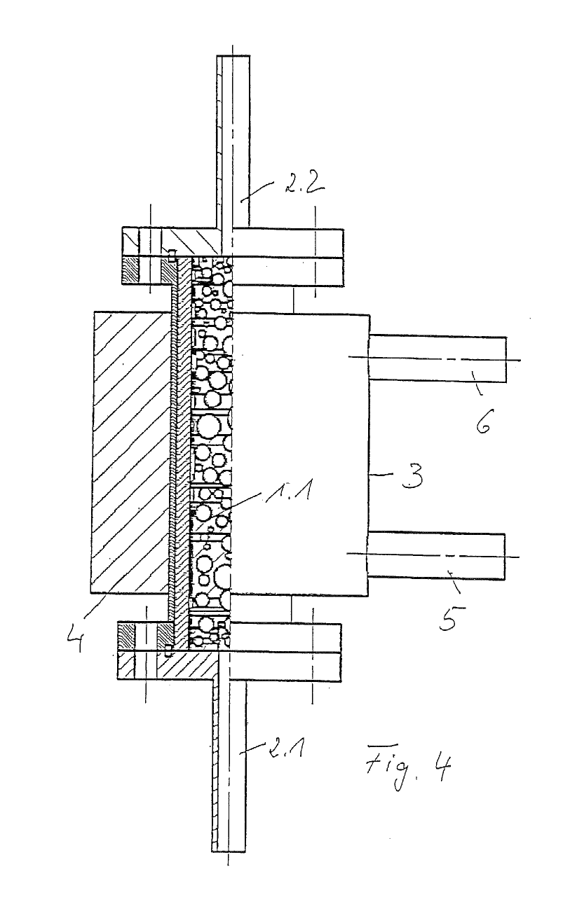

FIG. 4 a partial sectional representation through an example with an open-pore foam body through whose open pores the hydrocarbon compound or the liquid flows on the heating up to and above the boiling temperature.

A sectional representation is shown in FIG. 1 through a body 1 which has been obtained from a plurality of layers of an LTCC ceramic material connected to one another with material continuity by sintering. Sections have been removed in the individual layers which sections form a passage 1 conducted through the body 1 in a meandering manner. Ethanol or an ethanol-water mixture can flow into the passage 1 through the inlet 2.1 and can flow out of the outlet 2.2 again as a vapor/vapor mixture. The passage 2 has a cross-sectional surface of 1 mm of equal size over its total length. A tapering, at which the cross-sectional area of the passage 2 is reduced to 0.7 mm, is only formed at the outlet 2.2. It is adjoined by a conically formed widening with which a further homogenization of the pressure of the exiting vapor can be achieved over time. The passage 2 has a total length of 100 mm.

With a volume flow of 50 ml/h of supplied ethanol, the passage 2 was flowed through at a speed of 0.014 m/s. The heating took place with an energy of 11 W. Heating took place to a temperature above 100° C. to a maximum of 150° C. to evaporate the ethanol reliably and completely. The achievable pressure difference of the exiting vapor amounted to a maximum of 3 mbar over a longer time period so that the pressure fluctuation of the vapor to be taken into account for the following use can be neglected.

The heating took place via a twin-pipe jacket heating (not shown) through which hot gas was supplied at a temperature of at least 150° C. A corresponding heating of the body 1 and of the liquid to be evaporated can also take place using a further passage (likewise not shown) which is conducted through the body 1 next to the passage 2 and through which hot medium can be conducted for heating in counter-flow to the liquid to be evaporated. Alternatively, the heating can also take place using an electrical resistance heating. In this respect, electrical conductors can be provided through which an electrical current flows. Printed conductors, for example of silver, can be used for this purpose, for example. An electrical resistance heating can also be provided in combination with one of the possibilities explained above.

FIG. 2 shows an example in which, starting from an inlet 2.1 for the liquid to be evaporated, a passage 1 branches into a plurality of individual passages which are combined again in the direction of the outlet 2.2 in a body which has been produced from a ceramic material. In this respect, the liquid to be evaporated covers paths within the apparatus which are respectively of different lengths and remains in the apparatus for a correspondingly longer or shorter time. The pressure fluctuation over time of the exiting vapor can thereby also be reduced and be homogenized almost to a constant pressure.

This effect can likewise be utilized in an example such as is shown in FIG. 3. Starting from the inlet 2.2, a branching of the liquid to be evaporated also takes place here into a plurality of passages 2 which are combined again at the outlet 2.2.

The region around the outlet 2.2 can be configured in the examples in accordance with FIGS. 2 and 3 as in the example in accordance with FIG. 1 with tapering and widening.

An example with a body 1 which is configured as an open-pore foam body 1.1 is shown in FIG. 4. The hydrocarbon compound or the liquid can flow through the open pores of the foam body formed from SSiC on the heating up to and above the boiling temperature. It enters into the apparatus via the inlet 2.1, which is arranged vertically downwardly, flows through the foam body 1.1 and can then be supplied to a subsequent use in gaseous form via the vertically upwardly arranged outlet 2.2. The inlet 2.1 and the outlet 2.2 can be configured as simple pipes which are connected to the housing 3 via a flange connection, optionally also a weld connection. The hydrocarbon compound or the liquid can be introduced into the foam body 1.1 directly at the end of the inlet 2.1. There is also the possibility of providing a hollow space there in front of the foam body 1.1 in the flow direction, said hollow space having an enlarged cross-sectional area so that the flow speed is reduced and a homogenization and a uniform distribution of the hydrocarbon compound or of the liquid can be achieved before the evaporation which takes place within the foam body.

The foam body 1.1 in this example has a porosity of 80% to 90% and a pore density of 20 ppi.

In this example, a foam body 1.11.1 has been selected having a porosity which is constant within tight limits within the volume. There is, however, also the possibility of using one foam body 1.1 having a gradient porosity in the flow direction of the hydrocarbon compound or of the liquid or of using two foam bodies 1.1. having different porosities. In this respect, the porosity and/or pore size should increase in the flow direction of the hydrocarbon compound or of the liquid.

A housing 3 in which a hollow space 4 is present is formed in the foam body 1.1. In this example, a medium heated above the boiling temperature of the hydrocarbon compound or of the liquid can be led into the hollow space via the connector 5 and can be led off again via the connector 6. For this purpose, however, hot gas can also be used, in particular hot exhaust gas or exhaust air. The heating of the hydrocarbon compound or of the liquid in this respect takes place by heat exchange/recuperator.

In a non-illustrated form, however, an electrical resistance heating can also be arranged within the hollow space 4 with which the heating of the hydrocarbon compound or of the liquid up to and above the boiling temperature can be achieved.

In a specific experiment, ethanol having a volume flow of 50 ml/h was supplied via the inlet 2.1 having an inner diameter of 4 mm.

The foam body 1.1 had an outer diameter of 14 mm and a length of 70 mm in the flow direction of the hydrocarbon compound or of the liquid.

The ethanol was thereby heated to a temperature of at least 79° C. using an electrical resistance heating which was arranged around the foam body 1.1 and the vapor formed in this process was able to be led off for a further use at the outlet 2.2. The mean maximum pressure difference of the ethanol vapor exiting the outlet 2.2 was ±4 mbar.

### Reference Numeral List

1 body

1.1 foam body

2 passage

2.1 inlet

2.2 outlet

3 housing

4 hollow space

5 connector

6 connector

1. An apparatus for evaporating liquid hydrocarbon compounds or liquids in which hydrocarbon compounds are contained, wherein a heating apparatus is present at the apparatus by which a heating can be achieved above the boiling temperature of the respective hydrocarbon compound or of a liquid, characterized in that
the hydrocarbon compound or the liquid flows through at least one hollow space which is formed in a body or in a structure and the body or the structure is formed from a ceramic material which is inert for the respective hydrocarbon compound and/or for chemical compounds or chemical elements contained in the liquid.

2. An apparatus in accordance with claim 1, characterized in that a hollow space which is flowed through by the hydrocarbon compound or by the liquid is configured in the form of a passage.

3. An apparatus in accordance with claim 1, characterized in that a division has taken place within a body into at least two passages through which the hydrocarbon compound or liquid flows.

4. An apparatus in accordance with claim 1, characterized in that a passage or a plurality of passages are guided through the body in a meandering manner.

5. An apparatus in accordance with claim 1, characterized in that a plurality of passages conducted through the body have a different length and/or a different size of the cross-sectional surface.

6. An apparatus in accordance with claim 1, characterized in that a tapering of the free cross-sectional surface is present at the outlet of the one passage or of a plurality of passages, which is adjoined by a widening with an enlarged free cross-sectional surface.

7. An apparatus in accordance with claim 1, characterized in that at least one further passage is formed in the body, through which passage a hot medium with which a heating can be achieved to a temperature above the boiling temperature of the hydrocarbon compound is preferably conducted in cross-flow or in counter-flow.

8. An apparatus in accordance with claim 1, characterized in that the body is an open-pore foam body through whose open pores the hydrocarbon compound or the liquid flows on the heating up to and above the boiling temperature.

9. An apparatus in accordance with claim 1, characterized in that the porosity and/or the pore size within the foam body increases in the flow direction.

10. An apparatus in accordance with claim 1, characterized in that the hydrocarbon compound or the liquid flows through a structure formed from ceramic fibers.

11. An apparatus in accordance with claim 1, characterized in that a ceramic material is used for the manufacture of the body or of the structure which is selected from SiC, Si3N4, WC, MN, TiN and molybdenum silicide.

12. Use of the apparatus in accordance with claim 1 for the evaporation of ethanol or of an ethanol-water mixture for the operation of fuel cells, in particular of high-temperature fuel cells.
